SEQUENCE LISTING (1 sequence)

SEQ ID NO: 1 - protein, 309 aa, Bacillus sp.
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFTGGGSDGGTSP

(19) United States
(12) Patent Application Publication
(10) Pub. No.: US 2011/0091941 A1
(43) Pub. Date: Apr. 21, 2011

(54) Process For Producing Fermentation Products

(75) Inventor: Hans Sejr Olsen, Holte (DK)

(73) Assignee: Novozymes A/S, Bagsvaerd (DK)

(21) Appl. No.: 12/999,787

(22) PCT Filed: Jul. 3, 2009
(86) PCT No.: PCT/EP09/58459

§ 371 Date: Dec. 17, 2010

Related U.S. Application Data
(60) Provisional application No. 61/078,466, filed on Jul. 7, 2008.

(30) Foreign Application Priority Data
Jul. 3, 2008 (EP) 08159599.3

(51) Int. Cl. C12P 19/02; C12P 7/02

(52) U.S. Cl. 435/105; 435/155

(57) ABSTRACT

The invention relates to processes for hydrolyzing lignocellulose-containing material. The invention also relates to processes of producing fermentation products including a hydrolysis process of the invention.

### TECHNICAL FIELD

The present invention relates to processes for hydrolyzing lignocellulose-containing material and processes for producing fermentation products from lignocellulose-containing material.

### BACKGROUND OF THE INVENTION

Lignocellulose-containing feed stock can be hydrolyzed to glucose and other sugars. The sugars may used for producing fermentation products such as ethanol, e.g., for use as fuel. Producing fermentation products from lignocellulose is known in the art and generally includes pre-treating, hydrolyzing and fermenting the material.

The structure of lignocellulose is not directly accessible to enzymatic hydrolysis. Therefore, the lignocellulose is pre-treated in order to break the lignin seal and disrupt the crystalline structure of cellulose. This may cause solubilization and saccharification of the hemicellulose fraction. The cellulose fraction is then hydrolyzed enzymatically, e.g., by cellulolytic enzymes, which degrades the carbohydrate polymers into fermentable sugars. These fermentable sugars are then converted into the desired fermentation product by a fermenting organism, which product may optionally be recovered, e.g., by distillation.

Producing fermentation products from lignocellulose-containing material is currently very expensive. Consequently, there is a need for providing further processes for producing fermentation products from lignocellulose-containing materials.

### SUMMARY OF THE INVENTION

The present invention relates to processes of enzymatic pre-treatment and hydrolysis of lignocellulose-containing material and processes for producing fermentation products from lignocellulose-containing material.

In the first aspect the invention relates to a process for producing a hydrolysate comprising glucose, said process comprising; a) forming a slurry comprising lignocellulose-containing material and water, b) subjecting the lignocellulose-containing material to the action of one, several, or all of the enzyme activities protease, pectate lyase, ferulic acid esterase, and mannanase, at a pH of between 7 and 10, to loosen the cell wall structure and release cellulose fibrils, c) optionally isolating cellulose fibrils; d) subjecting the cellulose fibrils to the action an alkaline endo-glucanase composition at a pH of between 7 and 10, e) adjusting pH to between 4 and 7 and contacting the cellulose fibrils with a composition comprising cellulytic activities to obtain a hydrolyzate comprising glucose.

In an embodiment the hydrolyzate comprising glucose may be subjected to fermentation with a fermenting organism to produce a fermentation product, which optionally may be recovered.

### BRIEF DESCRIPTION OF DRAWINGS

FIG. 1 shows the comparison of the effect of enzymatic swelling on % conversion of cellulose to glucose.

FIG. 2 shows the main steps for production of ethanol from biomass by an alkaline process of the present invention.

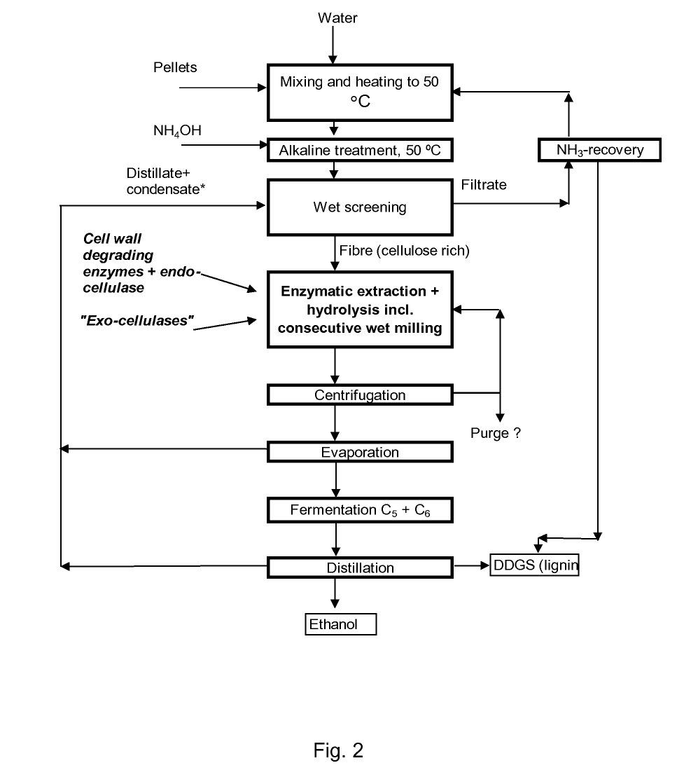

FIG. 3 shows the main steps for production of ethanol from biomass by an alkaline extraction process and a slightly acidic hydrolysis process of the present invention.

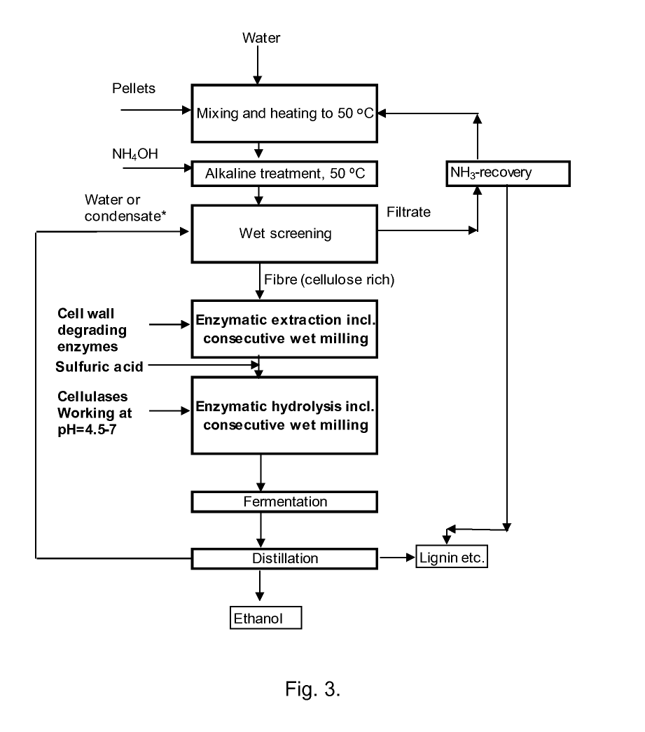

### DETAILED DESCRIPTION OF THE INVENTION

In the first aspect the invention relates to processes for hydrolyzing lignocellulose-containing materials.

The inventors have found that subjecting the lignocellulose-containing material to enzyme activities selected from protease and various cell wall degrading enzymes, i.e. hemi-cellulases, at a pH of between 7 and 10, and to the action of alkaline endo-glucanase at a pH of between 7 and 10 the cellulose of the lignocellulose-containing material can be made more accessible to the cellulytic enzymes activities applied for saccharification. Furthermore, the hydrolyzate produced has a low content of enzyme and/or yeast inhibitors.

### Lignocellulose-Containing Material

The term “lignocellulose-containing material” means material primarily consisting of cellulose, hemicellulose, and lignin. Lignocellulose-containing material is often referred to as “biomass”. Woody biomass is about 45-50% cellulose, 20-25% hemicellulose and 20-25% lignin. Herbaceous materials have lower cellulose, lower lignin and higher hemicellulose contents.

Cellulose is a linear beta 1->4 linked polymer of glucose. It is the principal component of all higher plant cell walls. In nature cellulose exists in crystalline and amorphous states. The thermodynamic stability of the beta 1->4 linkage and the capacity of cellulose to form internal hydrogen bonds gives it great structural strength. Cellulose is degraded to glucose through hydrolytic cleavage of the glycosidic bond.

Hemicellulose is a term used to refer to a wide variety of heteropolysaccharides found in association with cellulose and lignin in both woody and herbaceous plant species. The sugar composition varies with the plant species, but in angiosperms, the principal hemicellulosic sugar is xylose. Like cellulose, xylose occurs in the beta 1->4 linked backbone of the polymer. In gymnosperms, the principal component sugar is mannose. Arabinose is found as a side branch in some hemicelluloses.

Lignin is a phenylpropane polymer. Unlike cellulose and hemicellulose, lignin cannot be depolymerized by hydrolysis. Cleavage of the principal bonds in lignin require oxidation.

The lignocellulose-containing material may be any material containing lignocellulose. In a preferred embodiment the lignocellulose-containing material contains at least 30 wt.-%, preferably at least 50 wt.-%, more preferably at least 70 wt.-%, even more preferably at least 90 wt.-% lignocellulose. It is to be understood that the lignocellulose-containing material may also comprise other constituents such as proteinaceous material, starchy material, and sugars, such as fermentable sugars and/or un-fermentable sugars.

Lignocellulose-containing material is generally found, for example, in the stems, leaves, hulls, husks, and cobs of plants or leaves, branches, and wood of trees. Lignocellulose-containing material can also be, but is not limited to, herbaceous material, agricultural residues, forestry residues, municipal solid wastes, waste paper, and pulp and paper mill residues. It is to be understood that lignocellulose-containing material may be in the form of plant cell wall material containing lignin, cellulose and hemicellulose in a mixed matrix.

In a preferred embodiment the lignocellulose-containing material is corn fiber, rice straw, wheat bran, pine wood, wood chips, poplar, bagasse, paper and pulp processing waste.

Other examples include corn stover, corn fiber, hardwood, such as poplar and birch, softwood, cereal straw, such as, wheat straw, switch grass, Miscanthus, rice hulls, or mixtures thereof.

### Pre-Treatment

The lignocellulose-containing material may be pre-treated in any suitable way.

Pre-treatment is carried out before hydrolysis and/or fermentation. The goal of pre-treatment is to reduce the particle size, separate and/or release cellulose; hemicellulose and/or lignin and in this way increase the rate of hydrolysis. Pre-treatment processes such as wet-oxidation and alkaline pre-treatment targets lignin, while dilute acid and auto-hydrolysis targets hemicellulose. Steam explosion is an example of a pre-treatment that targets lignin.

The pre-treatment step may be a conventional pre-treatment step using techniques well known in the art. In a preferred embodiment pre-treatment takes place in a slurry of lignocellulose-containing material and water. The lignocellulose-containing material may during pre-treatment be present in an amount between 10-80 wt.-%, preferably between 20-70 wt.-%, especially between 30-60 wt.-%, such as around 50 wt-%.

Chemical, Mechanical and/or Biological Pre-Treatment

The lignocellulose-containing material may according to the invention be chemically, mechanically and/or biologically pre-treated before hydrolysis in accordance with the process of the invention. Mechanical pre-treatment (often referred to as “physical”—pre-treatment) may be carried out alone or may be combined with other pre-treatment processes.

Preferably, the chemical, mechanical and/or biological pre-treatment is carried out prior to the hydrolysis. Alternatively, the chemical, mechanical and/or biological pre-treatment may be carried out simultaneously with hydrolysis, such as simultaneously with addition of one or more hydrolyzing enzymes, and/or other enzyme activities, to release fermentable sugars, such as glucose and/or maltose.

### Chemical Pre-Treatment

The term “chemical pre-treatment” refers to any chemical pre-treatment which promotes the separation and/or release of cellulose, hemicellulose and/or lignin. Examples of suitable chemical pre-treatments include treatment with; for example, dilute acid, lime, alkaline, organic solvent, ammonia, sulfur dioxide, carbon dioxide. Further, wet oxidation and pH-controlled hydrothermolysis are also considered chemical pre-treatment.

Other pre-treatment techniques are also contemplated according to the invention. Cellulose solvent treatment has been shown to convert about 90% of cellulose to glucose. It has also been shown that enzymatic hydrolysis could be greatly enhanced when the lignocellulose structure is disrupted. Alkaline H2O2, ozone, organosolv (uses Lewis acids, FeCl3, (Al)2SO4 in aqueous alcohols), glycerol, dioxane, phenol, or ethylene glycol are among solvents known to disrupt cellulose structure and promote hydrolysis (Mosier et al. Bioresource Technology 96 (2005), p. 673-686).

Alkaline chemical pre-treatment with base, e.g., NaOH, Na2CO3 and/or ammonia or the like, is also within the scope of the invention. Pre-treatment processes using ammonia are described in, e.g., WO 2006/110891, WO 2006/11899, WO 2006/11900, WO 2006/110901, which are hereby incorporated by reference. Also the Kraft pulping process as described for example in “Pulp Processes” by Sven A. Rydholm, page 583-648. ISBN 0-89874-856-9 (1985) might be used. The solid pulp (about 50% by weight based on the dry wood chips) is collected and washed before the enzymatic treatments.

Wet oxidation techniques involve use of oxidizing agents, such as: sulphite based oxidizing agents or the like. Examples of solvent pre-treatments include treatment with DMSO (Dimethyl Sulfoxide) or the like. Chemical pre-treatment is generally carried out for 1 to 60 minutes, such as from 5 to 30 minutes, but may be carried out for shorter or longer periods of time dependent on the material to be pre-treated.

Other examples of suitable pre-treatment processes are described by Schell et al. (2003) Appl. Biochem and Biotechn. Vol. 105-108, p. 69-85, and Mosier et al. Bioresource Technology 96 (2005) 673-686, and US publication no. 2002/0164730, which references are hereby all incorporated by reference.

### Mechanical Pre-Treatment

The term “mechanical pre-treatment” refers to any mechanical (or physical) pre-treatment which promotes the separation and/or release of cellulose, hemicellulose and/or lignin from lignocellulose-containing material. For example, mechanical pre-treatment includes various types of milling, irradiation, steaming/steam explosion, and hydrothermolysis.

Mechanical pre-treatment includes comminution (mechanical reduction of the size). Comminution includes dry milling, wet milling and vibratory ball milling. Mechanical pre-treatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pre-treatment is carried out as a batch-process, in a steam gun hydrolyzer system which uses high pressure and high temperature as defined above. A Sunds Hydrolyzer (available from Sunds Defibrator AB (Sweden) may be used for this.

In a preferred embodiment the lignocellulose-containing material is subjected to a irradiation pre-treatment. The term “irradiation pre-treatment” refers to any pre-treatment by microwave e.g. as described by Zhu et al. “Production of ethanol from microwave-assisted alkali pre-treated wheat straw” in Process Biochemistry 41 (2006) 869-873 or ultrasonic pre-treatment, e.g., as described by e.g. Li et al. “A kinetic study on enzymatic hydrolysis of a variety of pulps for its enhancement with continuous ultrasonic irradiation”, in Biochemical Engineering Journal 19 (2004) 155-164.

### Combined Chemical and Mechanical Pre-Treatment

In a preferred embodiment the lignocellulose-containing material is subjected to both chemical and mechanical pre-treatment. For instance, the pre-treatment step may involve dilute or mild acid treatment and high temperature and/or pressure treatment. The chemical and mechanical pre-treatments may be carried out sequentially or simultaneously, as desired.

In a preferred embodiment the pre-treatment is carried out as a dilute and/or mild acid steam explosion step. In another preferred embodiment pre-treatment is carried out as an ammonia fiber explosion step (or AFEX pre-treatment step).

### Biological Pre-Treatment

The term “biological pre-treatment” refers to any biological pre-treatment which promotes the separation and/or release of cellulose, hemicellulose, and/or lignin from the lignocellulose-containing material. Known biological pre-treatment techniques involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh, P., and Singh, A., 1993, Physicochemical and biological treatments for enzymatic/microbial conversion of lignocellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolysates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95).

### Enzymatic Hydrolysis

Before the pre-treated lignocellulose-containing material is fermented it is hydrolyzed enzymatically to break down especially hemicellulose and/or cellulose into fermentable sugars.

According to the invention the enzymatic hydrolysis is performed in several steps. The lignocellulose-containing material to be hydrolyzed constitutes above 2.5% wt-%, preferably above 5% wt-%, preferably above 10% wt-%, preferably above 15 wt-% DS, preferably above 20 wt.-% DS, more preferably above 25 wt-% DS of the slurry of step a).

In step b) of the hydrolysis the lignocellulose-containing material is subjected to the action of one, or several or all enzyme activities selected from the group consisting of a protease, a pectate lyase, a ferulic acid esterase, and a mannanase. Also present may be any cellulytic enzyme, or hemi-cellulytic enzyme incl. any pectinolytic enzyme. Suitable are enzymes having activity above pH 7. The pH should be between 7 and 10, such as from 8 to 9, preferably around pH 8.5. The pH may be adjusted using NaOH, Ca(OH2) and/or KOH. The temperature may be between 20-70° C., preferably 30-60° C., and more preferably 40-55° C., e.g., around 50° C. During step b) the cell walls are degraded and the cellulose fibrils are made accessible for further hydrolysis. The hydrolysis in step b) may be carried out as a fed batch process where pre-treated lignocellulose-containing material is fed continuously/gradually or stepwise into a solution containing hydrolyzing enzymes.

In an embodiment a pectate lyase, a ferulic acid esterase, and a mannanase is present in the hydrolysis step (b). In an embodiment a pectate lyase, a ferulic acid esterase, mannanase and a cellulase is present. In an embodiment a pectate lyase, a ferulic acid esterase, mannanase, a cellulase and a protease is present.

In the optional step c) the cellulose fibrils may be isolated.

When the lignocellulose-containing material comprising very little lignin and/or hemicellulose e.g., such as municipal solid waste (MSW), industrial organic waste, office paper, waste paper, and pulp and paper mill residues or mixtures thereof steps b) and c) may be omitted.

In step d) of the hydrolysis the cellulose fibrils are treated with an alkaline endo-glucanase composition under neutral to basic pH conditions. In step d) the dry solids is preferably above 10 wt.-% DS, preferably above 15 wt-% DS, preferably above 20 wt.-% DS, more preferably above 25 wt-% DS.

The pH should be between 7 and 10, such as from 8 to 9, preferably around pH 8.5. The pH may be adjusted using NaOH, Ca(OH2) and/or KOH. The temperature may be between in range from 20-70° C., preferably 30-60° C., and more preferably 40-50° C. In an embodiment step e) and step f) are performed overlapping or simultaneously. The treatment during step d) causes a swelling of the cellulose fibrils thereby rupturing the crystalline structure making the cellulose more accessible for further hydrolysis.

In step e) of the hydrolysis the cellulose fibrils is treated with a cellulase composition comprising cellulolytic activity under neutral to acid pH conditions. Preferably the pH is between 4-7, preferably 5-7, such as around 5.5. The pH is preferably adjusted using phosphoric acid, succinic acid, hydrochloric acid and/or sulphuric acid. In step e) the dry solids is preferably above 10 wt.-% DS, preferably above 15 wt-% DS, preferably above 20 wt.-% DS, more preferably above 25 wt-% DS.

Preferably the temperature is in the range from 20-70° C., preferably 30-60° C., and more preferably 40-50° C.

Hydrolysis is typically carried out until the fermentable sugar yields are greater than 65%, preferably greater than 75%, more preferably greater than 85%.

The produced hydrolyzate has a high proportion of glucose resulting in a degree of fermentation of at least 60% after 24-120 hours of fermentation because of a low content of enzyme and yeast inhibitors.

### Fermentation

During the fermentation step f) the fermentable sugars from the hydrolyzed lignocellulose-containing material are fermented using one or more fermenting organisms capable of fermenting fermentable sugars, such as glucose, xylose, mannose, and galactose directly or indirectly into a desired fermentation product. The fermentation conditions depend on the desired fermentation product and can easily be determined by one of ordinary skill in the art.

In the case of ethanol fermentation with yeast the fermentation is preferably ongoing for between 5 and 120 hours, preferable 16 to 96 hours, more preferably between 24 and 72 hours. In an embodiment the fermentation is carried out at a temperature between 25° C. and 40° C., such as between 29° C. and 35° C., such as between 30° C. and 34° C., such as around 32° C. In an embodiment the pH is from pH 3-7, preferably 4-6.

In an embodiment the enzymatic hydrolysis continues after step e) and into the fermentation step f).

In a preferred embodiment the hydrolysis and fermentation is integrated in a Hybrid hydrolysis and fermentation (HHF). HHF begins with one or more separate hydrolysis step(s), where the lignocellulose is partly (e.g. 10-50%, such as 30% hydrolyzed) and ends with a simultaneous hydrolysis and fermentation step (SHF). The separate hydrolysis step is an enzymatic cellulose saccharification step typically carried out at conditions (e.g., at higher temperature) suitable, preferably optimal, for the hydrolyzing enzyme(s) in question. The subsequent simultaneous hydrolysis and fermentation step (SHF) is typically carried out at conditions suitable for the fermenting organism (often at lower temperature than the separate hydrolysis step).

In an embodiment the hydrolyzate is concentrated to 10% w/w-% dry matter before fermentation, preferably 15% w/w-% dry matter before fermentation. The hydrolyzate may be concentrated by evaporation or by a membrane process, e.g., reverse osmosis.

### Recovery

Subsequent to fermentation the fermentation product may optionally be separated from the fermentation medium in any suitable way. For instance, the medium may be distilled to extract the fermentation product or the fermentation product may be extracted from the fermentation medium by micro or membrane filtration techniques. Alternatively the fermentation product may be recovered by stripping. Recovery processes are well known in the art. The dry solids remaining after recovery comprising among other compounds lignin may be used in a boiler for steam and power production.

### Fermentation Products

The present invention may be used for producing any fermentation product. Preferred fermentation products include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H2 and CO2); antibiotics (e.g., penicillin and tetracycline); enzymes; vitamins (e.g., riboflavin, B12, beta-carotene); and hormones.

In a preferred embodiment the fermentation product is an alcohol, especially ethanol. The fermentation product, such as ethanol, obtained according to the invention, may preferably be used as fuel alcohol/ethanol. However, in the case of ethanol it may also be used as potable ethanol.

### Fermenting Organism

The term “fermenting organism” refers to any organism, including bacterial and fungal organisms, suitable for producing a desired fermentation product. Especially suitable fermenting organisms are able to ferment, i.e., convert, sugars, such as glucose, directly or indirectly into the desired fermentation product. Examples of fermenting organisms include fungal organisms, especially yeast. Preferred yeast includes strains of Saccharomyces spp., in particular strains of Saccharomyces cerevisiae or Saccharomyces uvarum; a strain of Pichia, preferably Pichia stipitis, such as Pichia stipitis CBS 5773; a strain of Candida, in particular a strain of Candida utilis, Candida diddensii, or Candida boidinii. Other fermenting organisms include strains of Zymomonas; Hansenula, in particular H. anomala; Klyveromyces, in particular K. fragilis; and Schizosaccharomyces, in particular S. pombe.

Commercially available yeast includes, e.g., ETHANOL RED™ yeast (available from Fermentis/Lesaffre, USA), FALI™ (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### Enzymes

Even if not specifically mentioned in context of a process or process of the invention, it is to be understood that the enzyme(s) (as well as other compounds) are used in an “effective amount”.

### Proteases

Any protease suitable for use under alkaline conditions can be used. Suitable proteases include those of animal, vegetable or microbial origin. Microbial origin is preferred. Chemically or genetically modified mutants are included. The protease may be a serine protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270.

Preferred commercially available protease enzymes include those sold under the trade names Everlase™, Kannase™, Alcalase™, Savinase™, Primase™, Durazym™, and Esperase™ by Novozymes A/S (Denmark), those sold under the tradename Maxatase, Maxacal, Maxapem, Properase, Purafect and Purafect OXP by Genencor International, and those sold under the tradename Opticlean and Optimase by Solvay Enzymes.

### Hemicellulolytic Enzymes

Any hemicellulase suitable for use in hydrolyzing hemicellulose, may be used. Preferred hemicellulases include pectate lyases, xylanases, arabinofuranosidases, acetyl xylan esterase, ferulic acid esterase, glucuronidases, endo-galactanase, mannases, endo or exo arabinases, exo-galactanses, and mixtures of two or more thereof. Preferably, the hemicellulase for use in the present invention is an endo-acting hemicellulase, and more preferably, the hemicellulase is an endo-acting hemicellulase which has the ability to hydrolyze hemicellulose under basic conditions of above pH 7, preferably pH 7-10.

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME® 200L, SHEARZYME® 500L, BIOFEED WHEAT®, and PULPZYME™ HC (from Novozymes) and GC 880, SPEZYME® CP (from Genencor Int).

The hemicellulase may be added in an amount effective to hydrolyze hemicellulose, such as, in amounts from about 0.001 to 0.5 wt.-% of total solids (TS), more preferably from about 0.05 to 0.5 wt.-% of TS.

Xylanases may be added in the amounts of 1.0-1000 FXU/kg dry solids, preferably from 5-500 FXU/kg dry solids, preferably from 5-100 FXU/kg dry solids and most preferably from 10-100 FXU/kg dry solids.

Xylanases may alternatively be added in amounts of 0.001-1.0 g/kg DM (dry matter) substrate, preferably in the amounts of 0.005-0.5 g/kg DM substrate, and most preferably from 0.05-0.10 g/kg DM substrate.

### Pectolytic Enzymes (or Pectinases)

Any pectinolytic enzyme that can degrade the pectin composition of plant cell walls may be used in practicing the present invention. Suitable pectinases include, without limitation, those of fungal or bacterial origin. Chemically or genetically modified pectinases are also encompassed. Preferably, the pectinase used in the invention are recombinantly produced and are mono-component enzymes.

Pectinases can be classified according to their preferential substrate, highly methyl-esterified pectin or low methyl-esterified pectin and polygalacturonic acid (pectate), and their reaction mechanism, beta-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by Enzyme Nomenclature (1992), e.g., pectate lyase (EC 4.2.2.2), pectin lyase (EC 4.2.2.10), polygalacturonase (EC 3.2.1.15), exo-polygalacturonase (EC 3.2.1.67), exo-polygalacturonate lyase (EC 4.2.2.9) and exo-poly-alpha-galacturonosidase (EC 3.2.1.82).

In embodiments the pectinase is a pectate lyase. Pectate lyase enzymatic activity as used herein refers to catalysis of the random cleavage of alpha-1,4-glycosidic linkages in pectic acid (also called polygalcturonic acid) by transelimination. Pectate lyases are also termed polygalacturonate lyases and poly(1,4-α-D-galacturonide) lyases.

The Pectate lyase (EC 4.2.2.2) is an enzyme which catalyse the random cleavage of α-1,4-glycosidic linkages in pectic acid (also called polygalacturonic acid) by transelimination. Pectate lyases also include polygalacturonate lyases and poly(1,4-α-D-galacturonide) lyases.

Examples of preferred pectate lyases are those that have been cloned from different bacterial genera such as Erwinia, Pseudomonas, Klebsiella, Xanthomonas and Bacillus, especially Bacillus licheniformis (U.S. Pat. No. 6,124,127), as well as from Bacillus subtilis (Nasser et al. (1993) FEBS Letts. 335:319-326) and Bacillus sp. YA-14 (Kim et al. (1994) Biosci. Biotech. Biochem. 58:947-949). Purification of pectate lyases with maximum activity in the pH range of 8-10 produced by Bacillus pumilus (Dave and Vaughn (1971) J. Bacteriol. 108:166-174), B. polymyxa (Nagel and Vaughn (1961) Arch. Biochem. Biophys. 93:344-352), B. stearothermophilus (Karbassi and Vaughn (1980) Can. J. Microbiol. 26:377-384), Bacillus sp. (Hasegawa and Nagel (1966) J. Food Sci. 31:838-845) and Bacillus sp. RK9 (Kelly and Fogarty (1978) Can. J. Microbiol. 24:1164-1172) have also been described.

A preferred pectate lyase may be obtained from Bacillus licheniformis as described in U.S. Pat. No. 6,124,127.

Other pectate lyases could be those that comprise the amino acid sequence of a pectate lyase disclosed in Heffron et al., (1995) Mol. Plant-Microbe Interact. 8: 331-334 and Henrissat et al., (1995) Plant Physiol. 107: 963-976.

A single enzyme or a combination of pectate lyases may be used. A preferred commercial pectate lyase preparation suitable for the invention is BioPrep® 3000 L available from Novozymes NS.

### Mannanases

In the context of the present invention a mannanase is a beta-mannanase and defined as an enzyme belonging to EC 3.2.1.78.

Mannanases have been identified in several Bacillus organisms. For example, Talbot et al., Appl. Environ. Microbiol., Vol. 56, No. 11, pp. 3505-3510 (1990) describes a beta-mannanase derived from Bacillus stearothermophilus having an optimum pH of 5.5-7.5. Mendoza et al., World J. Microbiol. Biotech., Vol. 10, No. 5, pp. 551-555 (1994) describes a beta-mannanase derived from Bacillus subtilis having an optimum activity at pH 5.0 and 55° C. JP-03047076 discloses a beta-mannanase derived from Bacillus sp., having an optimum pH of 8-10. JP-63056289 describes the production of an alkaline, thermostable beta-mannanase. JP-08051975 discloses alkaline beta-mannanases from alkalophilic Bacillus sp. AM-001. A purified mannanase from Bacillus amyloliquefaciens is disclosed in WO 97/11164. WO 94/25576 discloses an enzyme from Aspergillus aculeatus, CBS 101.43, exhibiting mannanase activity and WO 93/24622 discloses a mannanase isolated from Trichoderma reesei.

The mannanase may be derived from a strain of the genus Bacillus, such as the amino acid sequence having the sequence deposited as GENESEQP accession number AAY54122 and shown herein as SEQ ID NO:1 or an amino acid sequence which is homologous to this amino acid sequence.

A suitable commercial mannanase preparation is Mannaway® produced by Novozymes NS.

### Ferulic Esterases

In the context of the present invention a ferulic esterase is defined as an enzyme belonging to EC 3.1.1.73.

A suitable ferulic esterase preparation can be obtained from Malabrancea, e.g., from P. cinnamomea, such as e.g. a preparation comprising the ferulic esterase having the amino acid sequence shown in SEQ ID NO:2 in European patent application number 07121322.7, or an amino acid sequence which is homologous to this amino acid sequence.

Another suitable ferulic esterase preparation can be obtained from Penicillium, e.g., from P. aurantiogriseum, such as e.g. a preparation comprising the ferulic esterase having the amino acid sequence shown in SEQ ID NO:2 in European patent application number 0815469.7, or an amino acid sequence which is homologous to this amino acid sequence.

A suitable commercial ferulic esterase preparation preparation is Ultraflo produced by Novozymes NS.

### Alkaline Endo-Glucanases

The term “endoglucanase” means an endo-1,4-(1,3;1,4)-beta-D-glucan 4-glucanohydrolase (E.C. No. 3.2.1.4), which catalyses endo-hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Alkaline endo-glucanases are endo-glucanases having activity under alkaline conditions.

In a preferred embodiment endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In a preferred embodiment endoglucanases may be derived from a strain of the genus Bacillus akibai

In an embodiment the alkaline endo-glucanase composition is one of the commercially available products CAREZYME®, ENDOLASE® and CELLUCLEAN® (Novozymes NS, Denmark). The enzyme may be applied in a dosage of 1-100 g/kg cellulose.

### Acid Cellulolytic Activity

The term “acid cellulolytic activity” as used herein are understood as comprising enzymes having cellobiohydrolase activity (EC 3.2.1.91), e.g., cellobiohydrolase I and/or cellobiohydrolase II, as well as endo-glucanase activity (EC 3.2.1.4) and/or beta-glucosidase activity (EC 3.2.1.21) having activity at pH below 6.

The cellulolytic activity may, in a preferred embodiment, be in the form of a preparation of enzymes of fungal origin, such as from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In preferred embodiment the cellulolytic enzyme preparation contains one or more of the following activities: endoglucanase, cellobiohydrolases I and II, and beta-glucosidase activity.

In a preferred embodiment cellulolytic enzyme preparation is a composition disclosed in WO2008/151079, which is hereby incorporated by reference. In a preferred embodiment the cellulolytic enzyme preparation comprising a polypeptide having cellulolytic enhancing activity, preferably a family GH61A polypeptide, preferably those disclosed in WO 2005/074656 (Novozymes). The cellulolytic enzyme preparation may further comprise beta-glucosidase, such as beta-glucosidase derived from a strain of the genus Trichoderma, Aspergillus or Penicillium, including the fusion protein having beta-glucosidase activity disclosed in co-pending application U.S. 60/832,511 (Novozymes). In a preferred embodiment the cellulolytic enzyme preparation may also comprises a CBH II enzyme, preferably Thielavia terrestris cellobiohydrolase II (CEL6A). In another preferred embodiment the cellulolytic enzyme preparation may also comprise cellulolytic enzymes; preferably those derived from Trichoderma reesei or Humicola insolens.

The cellulolytic enzyme composition may also comprising a polypeptide having cellulolytic enhancing activity (GH61A) disclosed in WO 2005/074656; a beta-glucosidase (e.g., fusion protein disclosed in U.S. 60/832,511 and PCT/US2007/074038), and cellulolytic enzymes derived from Trichoderma reesei. The cellulolytic enzyme composition

In another preferred embodiment the cellulolytic composition comprising a polypeptide having cellulolytic enhancing activity (GH61A) disclosed in WO 2005/074656; a beta-glucosidase (e.g., fusion protein disclosed in U.S. 60/832,511 and PCT/US2007/074038), Thielavia terrestris cellobiohydrolase II (CEL6A), and cellulolytic enzymes preparation derived from Trichoderma reesei.

In an embodiment the cellulolytic enzyme composition is the commercially available product CELLUCLAST™ 1.5L, CELLUZYME™ (Novozymes A/S, Denmark) or ACCELLARASE™ 1000 (Genencor Int, Inc., USA).

The cellulolytic activity may be dosed in the range from 0.1-100 FPU per gram total solids (TS), preferably 0.5-50 FPU per gram TS, especially 1-20 FPU per gram TS.

### Cellulolytic Enhancing Activity

The term “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material, e.g., pre-treated lignocellulose-containing material by cellulolytic protein under the following conditions: 1-50 mg of total protein/g of cellulose in PCS (pre-treated corn stover), wherein total protein is comprised of 80-99.5% w/w cellulolytic protein/g of cellulose in PCS and 0.5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide. WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Published Application Serial No. 2007/0077630 discloses an isolated polypeptide having cellulolytic enhancing activity and a polynucleotide thereof from Trichoderma reesei.

### Alpha-Amylase

According to the invention any alpha-amylase may be used, such as of fungal, bacterial or plant origin. In a preferred embodiment the alpha-amylase is an acid alpha-amylase, e.g., acid fungal alpha-amylase or acid bacterial alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylase

According to the invention a bacterial alpha-amylase is preferably derived from the genus Bacillus.

In a preferred embodiment the Bacillus alpha-amylase is derived from a strain of Bacillus licheniformis, Bacillus amyloliquefaciens, Bacillus subtilis or Bacillus stearothermophilus, but may also be derived from other Bacillus sp. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/19467 (all sequences hereby incorporated by reference). In an embodiment the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted 1181*+G182*+N193F) compared to the wild-type BSG alpha-amylase amino acid sequence set forth in SEQ ID NO:3 disclosed in WO 99/19467.

In an embodiment the bacterial alpha-amylase is dosed in an amount of 0.0005-5 KNU per g DS, preferably 0.001-1 KNU per g DS, such as around 0.050 KNU per g DS.

### Fungal Alpha-Amylase

Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e. at least 70%, at least 75%, at least 80%, at least 85%, at least 90%, at least 95%, at least 96%, at least 97%, at least 98%, at least 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3—incorporated by reference). A commercially available acid fungal alpha-amylase derived from Aspergillus niger is SP288 (available from Novozymes NS, Denmark).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al. J. Ferment. Bioeng. 81:292-298 (1996) “Molecular-cloning and determination of the nucleotide-sequence of a gene encoding an acid-stable alpha-amylase from Aspergillus kawachii.”; and further as EMBL:#AB008370.

The fungal alpha-amylase may also be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

An acid alpha-amylases may according to the invention be added in an amount of 0.001 to 10 AFAU/g DS, preferably from 0.01 to 5 AFAU/g DS, especially 0.3 to 2 AFAU/g DS or 0.001 to 1 FAU-F/g DS, preferably 0.01 to 1 FAU-F/g DS.

### Commercial Alpha-Amylase Products

Preferred commercial compositions comprising alpha-amylase include MYCOLASE™ from DSM (Gist Brocades), BAN™, TERMAMYL™ SC, FUNGAMYL™, LIQUOZYME™ X, LIQUOZYME™ SC and SAN™ SUPER, SAN™ EXTRA L (Novozymes A/S) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes NS, Denmark).

### Carbohydrate-Source Generating Enzyme

The term “carbohydrate-source generating enzyme” includes glucoamylase (being glucose generators), beta-amylase and maltogenic amylase (being maltose generators) and also pullulanase and alpha-glucosidase. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated mixtures are mixtures of at least a glucoamylase and an alpha-amylase, especially an acid amylase, even more preferred an acid fungal alpha-amylase. The ratio between acid fungal alpha-amylase activity (FAU-F) and glucoamylase activity (AGU) (i.e., FAU-F per AGU) may in an embodiment of the invention be between 0.1 and 100, in particular between 2 and 50, such as in the range from 10-40.

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka, Y. et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. thermohydrosulfuricum (WO 86/01831) and Trametes cingulata, Pachykytospora papyracea; and Leucopaxillus giganteus all disclosed in WO 2006/069289; or Peniophora rufomarginata disclosed in PCT/US2007/066618; or a mixture thereof. Also hybrid glucoamylase are contemplated according to the invention. Examples the hybrid glucoamylases disclosed in WO 2005/045018. Specific examples include the hybrid glucoamylase disclosed in Table 1 and 4 of Example 1 (which hybrids are hereby incorporated by reference).

Contemplated are also glucoamylases which exhibit a high identity to any of above mention glucoamylases, i.e., at least 70%, at least 75%, at least 80%, at least 85%, at least 90%, at least 95%, at least 96%, at least 97%, at least 98%, at least 99% or even 100% identity to the mature enzymes sequences mentioned above.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes NS); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.0001-20 AGU/g DS, preferably 0.001-10 AGU/g DS, especially between 0.01-5 AGU/g DS, such as 0.1-2 AGU/g DS.

### Materials & Methods

### Determination of Identity

The relatedness between two amino acid sequences or between two nucleotide sequences is described by the parameter “identity”.

The degree of identity between two amino acid sequences may be determined by the Clustal process (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=1, gap penalty=3, windows=5, and diagonals=5.

The degree of identity between two nucleotide sequences may be determined by the Wilbur-Lipman process (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

### Cellulase Activity Using Filter Paper Assay (FPU Assay)

### 1. Source of Process

1.1 The process is disclosed in a document entitled “Measurement of Cellulase Activities” by Adney, B. and Baker, J. 1996. Laboratory Analytical Procedure, LAP-006, National Renewable Energy Laboratory (NREL). It is based on the IUPAC process for measuring cellulase activity (Ghose, T. K., Measurement of Cellulse Activities, Pure & Appl. Chem. 59, pp. 257-268, 1987.

### 2. Procedure

2.1 The process is carried out as described by Adney and Baker, 1996, supra, except for the use of a 96 well plates to read the absorbance values after color development, as described below.

### 2.2 Enzyme Assay Tubes:

2.2.1 A rolled filter paper strip (#1 Whatman; 1×6 cm; 50 mg) is added to the bottom of a test tube (13×100 mm).
2.2.2 To the tube is added 1.0 mL of 0.05 M Na-citrate buffer (pH 4.80).
2.2.3 The tubes containing filter paper and buffer are incubated 5 min. at 50° C. (±0.1° C.) in a circulating water bath.
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds.
2.2.6 After vortexing, the tubes are incubated for 60 mins. at 50° C. (±0.1° C.) in a circulating water bath.
2.2.7 Immediately following the 60 min. incubation, the tubes are removed from the water bath, and 3.0 mL of DNS reagent is added to each tube to stop the reaction. The tubes are vortexed 3 seconds to mix.

### 2.3 Blank and Controls

2.3.1 A reagent blank is prepared by adding 1.5 mL of citrate buffer to a test tube.
2.3.2 A substrate control is prepared by placing a rolled filter paper strip into the bottom of a test tube, and adding 1.5 mL of citrate buffer.
2.3.3 Enzyme controls are prepared for each enzyme dilution by mixing 1.0 mL of citrate buffer with 0.5 mL of the appropriate enzyme dilution.
2.3.4 The reagent blank, substrate control, and enzyme controls are assayed in the same manner as the enzyme assay tubes, and done along with them.

### 2.4 Glucose Standards

2.4.1 A 100 mL stock solution of glucose (10.0 mg/mL) is prepared, and 5 mL aliquots are frozen. Prior to use, aliquots are thawed and vortexed to mix.
2.4.2 Dilutions of the stock solution are made in citrate buffer as follows:
G1=1.0 mL stock+0.5 mL buffer=6.7 mg/mL=3.3 mg/0.5 mL
G2=0.75 mL stock+0.75 mL buffer=5.0 mg/mL=2.5 mg/0.5 mL
G3=0.5 mL stock+1.0 mL buffer=3.3 mg/mL=1.7 mg/0.5 mL
G4=0.2 mL stock+0.8 mL buffer=2.0 mg/mL=1.0 mg/0.5 mL
2.4.3 Glucose standard tubes are prepared by adding 0.5 mL of each dilution to 1.0 mL of citrate buffer.
2.4.4 The glucose standard tubes are assayed in the same manner as the enzyme assay tubes, and done along with them.

### 2.5 Color Development

2.5.1 Following the 60 min. incubation and addition of DNS, the tubes are all boiled together for 5 mins. in a water bath.
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH2O in a 96-well plate. Each well is mixed, and the absorbance is read at 540 nm.

### 2.6 Calculations (Examples are Given in the NREL Document)

2.6.1 A glucose standard curve is prepared by graphing glucose concentration (mg/0.5 mL) for the four standards (G1-G4) vs. A540. This is fitted using a linear regression (Prism Software), and the equation for the line is used to determine the glucose produced for each of the enzyme assay tubes.
2.6.2 A plot of glucose produced (mg/0.5 mL) vs. total enzyme dilution is prepared, with the Y-axis (enzyme dilution) being on a log scale.
2.6.3 A line is drawn between the enzyme dilution that produced just above 2.0 mg glucose and the dilution that produced just below that. From this line, it is determined the enzyme dilution that would have produced exactly 2.0 mg of glucose.
2.6.4 The Filter Paper Units/mL (FPU/mL) are calculated as follows:
FPU/mL=0.37/enzyme dilution producing 2.0 mg glucose

### Xylose/Glucose Isomerase Assay (IGIU)

1 IGIU is the amount of enzyme which converts glucose to fructose at an initial rate of 1 micromole per minute at standard analytical conditions.

Standard Conditions:

Glucose concentration: 45% w/w

pH: 7.5

Temperature: 60° C.

Mg2+ concentration: 99 mg/l (1.0 g/l MgSO4*7H2O)

Ca2+ concentration <2 ppm

Activator, SO2 concentration: 100 ppm (0.18 g/l Na2S2O5)

Buffer, Na2CO3, concentration: 2 mM Na2CO3

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties. The present invention is further described by the following examples which should not be construed as limiting the scope of the invention.

### Cellulytic Activity (EGU)

The cellulytic activity may be measured in endo-glucanase units (EGU), determined at pH 6.0 with carboxymethyl cellulose (CMC) as substrate.

A substrate solution is prepared, containing 34.0 g/l CMC (Hercules 7 LFD) in 0.1 M phosphate buffer at pH 6.0. The enzyme sample to be analyzed is dissolved in the same buffer. 5 ml substrate solution and 0.15 ml enzyme solution are mixed and transferred to a vibration viscosimeter (e.g. MIVI 3000 from Sofraser, France), thermostated at 40° C., for 30 minutes.

One EGU is defined as the amount of enzyme that reduces the viscosity to one half under these conditions. The amount of enzyme sample should be adjusted to provide 0.01-0.02 EGU/ml in the reaction mixture.

### Mannanase Activity (MIUM)

One MIUM is defined as the activity of one mg of pure enzyme protein of SEQ ID NO:1 herein. The assay is performed at Temp=30° C., Buffer, 0.1 M Tris pH 7.75, 4.5% PEG 6000.

### Pectate Lyase Activity (APSU)

Pectate Lyase catalyses the formation of double bonds in polygalacturonic acid. The number of formed double bonds is determined by photometric measurement at 235 nm. One APSU (Alcalophile Pectate Lyase Unit) is defined as the amount of enzyme that produces C═C double bonds equivalent to 1 μmol unsaturated digalacturonic acid per minute under the standard conditions:

Temperature: 37.0° C.±0.5° C.

pH: 10.00±0.05

Wavelength: 235 nm in a 1 cm cuvette

Incubation time: 10 min.

Time of Measurement: 30 min.

Enzyme concentration range: 0.05-0.15 APSU/mL

Limit of quantification: 1.25 APSU/g

Range: [50; 150] mAPSU/mL

### Other Processes

Dry matter: Mettler Toledo HR 73 Halogen Moisture dryer
BRIX: RFM830 Digital refractometer from Bilingham & Stanley Ltd.
pH: WTW pH-meter
Milling: “coffee” grinder Bosch type KM13 (E nr: MKM 6003 FD 9512) for 2 minutes
HPLC: Waters 717 Autosampler, Waters 515 Pump and a Waters 2414 Refractive index detector. A column type Bio-rad (Animex HPX-87 H 300-7.8 mm), Cat no. 125140 was used. Standards were used for glucose, maltose, maltotriose, xylose, and maltotetraose

### Enzymes Used in the Examples:

A pectate lyase (EC 4.2.2.2) preparation derived from a Bacillus sp. and available from Novozymes as BioPrep® 3000 L with an activity of 3000 APSU/g composition.

An endo-xylanase (EC 3.2.1.8) composition derived from Bacillus agaradhaerens and available from Novozymes as Pulpzyme® HC.

Cellulase composition A comprising acid cellulolytic enzymes derived from Trichoderma reesei, a GH61A polypeptide disclosed in WO2005/074656, and an Aspergillus oryzae beta-glucosidase (in the fusion protein disclosed in WO2008/057637). Cellulase composition A is disclosed in WO2008/151079. Cellulase composition A has an activity 180 FPU/g composition.

Cellulase composition B comprising alkaline endo-cellulase derived from Bacillus sp. and available from Novozymes as Celluclean® Conc. with an activity of 320000 ECU/g composition.

Ferulic acid esterase composition also comprising alkaline cellulase. The composition is derived from Humicola insolens and available from Novozymes as Novozym® 342 with an activity of 90 EGU/g

Mannanase (EC 3.2.1.25) composition comprising a mannanase having the amino acid sequence shown in SEQ ID NO:1 herein and with an activity of 40 MIUM/g composition.

### Example 1

Lignocellulosic material was washed under alkaline conditions to remove soluble compounds of the lignin and to swell the remaining material. The soluble compounds removed during the washing include enzyme inhibitors. The enzymatic pre-treatment with cell wall degrading enzymes performed during washing opens the recalcitrant structure of the biomass and render the cellulose part of the material more accessible to hydrolysis by cellulases.
1. Biomass in the form of 200 g wheat straw was milled and a slurry of the milled straw in 2000 mL of 1.2% NaOH was prepared by slow stirring for 2 h at room temperature.
2. The material was purred onto a screen having a mesh size of 0.295 mm and washed using 30 L of tap water. Excess water was removed by pressing. The dry matter content of the press cake was determined to 9.4% w/w and pH was measured to 8.3.
3. 2000 g slurry having 6.7% w/w dry matter was prepared and 4 reactors loaded with 500 g in each, and placed in a water bath at 50° C.
4. Pectate lyase (15 APSU), endo-xylanase (0.03 mg enzyme protein/g washed biomass), ferulic acid esterase composition with alkaline cellulase (0.45 EGU), mannose (0.20 MIUM) were applied per g of washed biomass.
5. Samples of 10 mL were drawn at 0; 10; 60; 120 and 180 minutes.
6. The samples were analyzed for pH directly. The 10 mL samples were centrifuged for 10 minutes at 3800×G (G is gravity) in a graduated tube. The percentage of solid phase V/V was determined.
7. The supernatant was analyzed by HPLC for soluble sugars and oligosaccharides, and % dry matter was determined.

TABLE 1
Solid phase volume, supernatant dry matter during the reaction.

| Reaction time, | % solid | Supernatant % | Σ of soluble | |
|---|---|---|---|---|
| minutes | phase | dry matter | sugars (g/L) | pH |
| 0 | 55 | 0.20 | 6.2 | n.a. |
| 10 | 15 | 0.20 | 3.3 | n.a. |
| 60 | 20 | 0.53 | 4.9 | n.a. |
| 120 | 25 | 1.05 | 7.5 | n.a. |
| 180 | 20 | 1.98 | 14.8 | 8.2 |

TABLE 2
Composition of soluble sugars in g/L after 180 minutes reaction.

| DP1 + DP2 | DP3 | DP4 | C5 | Oligosacch. | Σ |
|---|---|---|---|---|---|
| 0.1 | 1.5 | 11.3 | 0.1 | 1.8 | 14.8 |

C5 is 5-carbon sugars and primarily xylose.

### Example 2

Example 2, illustrates the effect of the cellulose swelling under alkaline conditions on the subsequent hydrolysis.
1. In a reaction flask equipped with stirrer 25 g of cellulose (Cellulose microcrystalline colloidal—SigmaAldrich product no. 435244) was mixed with 450 g of 0.15 N NaHCO₃. pH was adjusted to 8.5 and the temperature to 50° C.
2. Cellulase composition B comprising alkaline endo-cellulase in an amount equivalent to 350 ECU/g cellulose was added to the flask.
3. A reaction was carried out under stirring for 60 minutes.
4. pH was adjusted to 5.0-5.2 (aimed at pH=5.0) by addition of dry succinic acid.
5. Acid cellulase composition A in an amount equivalent to 4 FPU/g cellulose was added to the flask.
6. During the reaction samples were taken for measurement of solid phase volume after centrifugation of a 10 mL sample at 3800 rpm (˜3000×G) for 10 minutes and HPLC was carried out for determination of the content of glucose on the supernatants.

### Control Treatment.

1. To a reaction flask equipped with stirrer 25 g of cellulose was mixed with 450 g of 0.15 N NaHCO₃. pH was adjusted to 8.5 and the flask was incubated at 50° C.
2. A reaction was carried out under stirring for 60 minutes.
3. 3 to 6 were performed as above.

TABLE 3
Effect of pretreatment with endo-cellulase

| | Pre-treated with endo- | | | | | | | |
|---|---|---|---|---|---|---|---|---|
| | cellulase | | | | Control treatment. | | | |
| Time, hours | 0 | 21 | 44 | 75 | 0 | 21 | 44 | 75 |
| Solid phase | 26 | 46 | 35 | 20 | 26 | 62 | 50 | 37 |
| volume % | | | | | | | | |
| Glucose g/L | 0.03 | 13.7 | 19.7 | 25.3 | 0.03 | 11.6 | 16.9 | 21.5 |

### Example 3

This example combines the steps of example 1 and example 2. The washing process carried out under alkaline conditions removes parts of the lignin and swell the recalcitrant components of the biomass material. The enzymatic pre-treatment with cell wall degrading enzymes under alkaline conditions leaves the cellulose in a form that is readily hydrolysed by use of the acid cellulases.
1. 2.5 kg pelleted wheat straw (fuel pills) was suspended in 22.5 litre of tap water at 50° C. and NaOH was added to a final concentration of 1.2%. The slurry was incubated with stirring for 2 hours at 50° C.
2. A wet screening was performed using an Algaier VTS 600 vibrating tumbler screen with a 40 microM mesh screen and the solid phase was collected.
3. The solid phase was washed using 100 litre of water at 50° C. and re-screened. This procedure was repeated until pH was about 8.5.
4. From the filtrate the remaining solids was recovered using a Westfalia separator type SB7 and the collected solids was added to the screened solids. The combined product is called pulp in the following. The pulp could be is pumped.
5. The pulp was recirculated through a toothed colloid mill (Fryma Mill type MZ 80) for 30 minutes. The cell wall degrading enzyme activities was dosed as in Example 1 table 1.
6. The reaction was carried out over a total period of 4 hours with consecutive recirculation every 30 minutes.
7. 500 g of the slurry produced as above was placed in a reaction flask equipped with stirrer and thermostated to 50° C. in a water bath. pH was 8.5.
8. The cellulose content in the slurry was determined using the process as described by R. Sun et al. Industrial Crops and Products 4 (1995) 127-145.
9. Cellulase composition B in an amount of 350 ECU/g cellulose was dosed.
10. A reaction was carried out under stirring for 60 minutes.
11. pH was adjusted to 5.0 by addition of dry succinic acid.
12. An enzyme dosage of Cellulase composition A in an amount equivalent to 3.4 FPU/g dry matter cellulose was added to the flask.
13. During the reaction samples were taken for determination of the content of glucose on the superanatants by HPLC. The % conversion of cellulose was determined as the yield of glucose in the clear supernatant in relation to the theoretical glucose content based on the mass of 100% hydrolysed cellulose.

### Control.

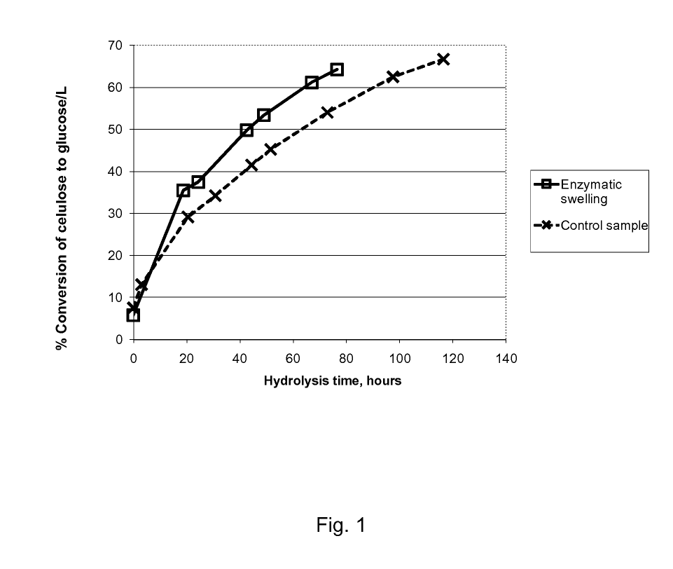

1. 500 g of the slurry after step 6 was placed in a reaction flask equipped with stirring. pH was adjusted to 5.0 by addition of dry succinic acid.
2. An enzyme dosage of Cellulase composition A in an amount equivalent to 3.8 FPU/g dry matter was added to the flask.
3. During the reaction samples were taken for determination of the content of glucose on the supernatants by HPLC. The % conversion of cellulose was determined as in Example 1. The results are shown in FIG. 1.

1-22. (canceled)

23. A process for producing a hydrolysate comprising glucose, said process comprising:
a) forming a slurry comprising lignocellulose-containing material and water,
b) subjecting the lignocellulose-containing material to the action of one, several, or all of the enzyme activities protease, pectate lyase, ferulic acid esterase, and mannanase, at a pH of between 7 and 10, to loosen the cell wall structure and release cellulose fibrils,
c) optionally isolating cellulose fibrils;
d) subjecting the cellulose fibrils to the action an alkaline endoglucanase composition at a pH of between 7 and 10; and
e) adjusting the pH to between 4 and 7 and contacting the cellulose fibers with a composition comprising cellulytic activities to obtain a hydrolyzate comprising glucose.

24. The process of claim 23, further comprising,
f) subjecting the glucose to fermentation with a fermenting organism to produce a fermentation product, and
g) optionally recovering the fermentation product.

25. The process of claim 23, wherein the lignocellulose-containing material content is adjusted by continuous or stepwise addition of lignocellulose-containing material to the slurry.

26. The process of claim 23, wherein the lignocellulose-containing material constitutes above 2.5% wt. % DS of the slurry of step a).

27. The process of claim 23, wherein the hydrolysis in step b) is performed in the presence of a pectate lyase, a ferulic acid esterase, and a mannanase.

28. The process of claim 23, wherein also a cellulase is present.

29. The process of claim 23, wherein also a protease is present.

30. The process of claim 23, wherein also an alpha-amylase is present.

31. The process of claim 23, wherein the hydrolysis step a) and/or c) is carried out at a pH in the range from 8 to 9.

32. The process of claim 23, wherein the hydrolysis step(s) b), d) and/or e) is carried out at a temperature in the range from 20-70° C.

33. The process of claim 23, wherein the hydrolysis step e) is carried out at a pH between 4-7.

34. The process of claim 23, wherein the hydrolysis steps b) and c) are performed simultaneously.

35. The process of claim 23, wherein the pH following step c) is adjusted using NaOH, Ca(OH)₂ and/or KOH

36. The process of claim 23, wherein the pH following step e) is adjusted using phosphoric acid, succinic acid, hydrochloric acid and/or sulphuric acid.

37. The process of claim 23, wherein the biomass prior to step a) has been subjected to a pretreatment comprising microwave and/or ultrasonic irradiation treatment

38. The process of claim 23, wherein the lignocellulose-containing material is chemically, mechanical and/or biologically pre-treated before enzymatic hydrolysis.

39. The process of claim 23, wherein further one or more starch-degrading enzymes, such as alpha-amylases and/or glucoamylases, are present during hydrolysis or hydrolysis and fermentation.

40. The process of claim 23, wherein the fermentation product is an alcohol.

41. The process of claim 23, wherein the fermenting organism is yeast.

42. The process of claim 23, wherein the lignocellulose-containing material is derived from corn stover, corn fiber, hard wood, such as poplar and birch, softwood, cereal straw, such as, wheat straw, switch grass, Miscanthus, rice hulls, municipal solid waste (MSW), industrial organic waste, office paper, or mixtures thereof.
